(19) United States
(12) Patent Application Publication
(10) Pub. No.: US 2012/0141434 A1
(43) Pub. Date: Jun. 7, 2012

(54) Methods for Selecting Expanded Stem Cell Populations

(75) Inventors: Tony Peled, Mevasseret Zion (IL); Dorit Harati, Jerusalem (IL)

(73) Assignee: Gamida Cell Ltd., Jerusalem (IL)

(21) Appl. No.: 13/319,157

(22) PCT Filed: May 5, 2010
(86) PCT No.: PCT/IL2010/000366

§ 371 Date: Feb. 17, 2012

Related U.S. Application Data
(60) Provisional application No. 61/176,153, filed on May 7, 2009.

(51) Int. Cl. A61K 35/12; C12Q 1/06; A61P 35/00; C12N 5/0789; A61P 7/00; A61P 35/02; G01N 33/566; C12Q 1/02

(52) U.S. Cl. 424/93.7; 435/7.21; 435/39; 435/29; 435/325

(57) ABSTRACT

A method and criteria are provided for selecting expanded hematopoietic stem cell populations for allogenic transplantation, the selected cell populations having high probability of engraftment and positive clinical outcome.

### FIELD AND BACKGROUND OF THE INVENTION

The present invention relates to a method of selecting populations of hematopoietic cells for transplantation to subjects in need thereof. Hematopoietic cell populations are expanded ex-vivo, and selected according to predetermined criteria, to produce populations of expanded hematopoietic cells with a high probability of engraftment. The method may further comprise selecting populations of cells for ex-vivo expansion. The invention also relates to the therapeutic use of the selected populations for transplantation in the clinical setting, for treatment of blood disorders, traumatic injuries and cancer.

Cord blood, bone marrow and other hematopoietic-rich tissues are a valuable source of stem cells, particularly where a matched unrelated donor cannot be found within a reasonable time. Advantages of the use of cord blood include the fact that it is readily available, carries less risk of transmission of blood-borne infectious diseases, and is transplantable across HLA barriers with diminished risk of graft-versus-host disease compared with similarly mismatched stem cells from the peripheral blood or bone marrow of related or unrelated donors. Another important advantage of cord blood is the rapidity with which an acceptable cord-blood unit, once identified, can be acquired.

However, a major clinical limitation of hematopoietic cell transplantation is the low number of hematopoietic stem/progenitor cells available from sources such as mobilized peripheral blood, bone marrow grafts and cord blood. Even though cord blood has a higher percentage of progenitor cells than adult bone marrow, the fixed cell content that is ultimately derived from cord blood represents a major challenge, with cord blood having the lowest numbers of hematopoietic stem/progenitor cells of all sources. Transplanted cord blood cells also have been shown to have a slower time to engraftment and higher rate of engraftment failure, although functionally, human cord blood-derived cells appear to engraft immunodeficient mice better with fewer cell numbers than bone marrow- or peripheral blood-derived cells. CD34+ cell dose greater 1.7×10⁵ CD34⁺ cells/kg has been the one factor consistently identified as significantly associated with rate of engraftment, time to neutrophil engraftment and survival. Transplant related mortality was 20% for patients who received greater than 1.7×105 CD34+ cells/kg versus 75% for patients that received a cell dose below this threshold. Thus, it has been postulated that outcomes could be improved by performing transplantation with the currently recommended cell dose and HLA disparity (more than 2×10⁷ nucleated cells per kilogram of the recipient's body weight and two or fewer of six HLA mismatches, respectively).

Indeed, in cases where a suitable cord blood unit has been identified, and either an urgent transplant is indicated, or a suitable bone marrow unit is unavailable, recently developed clinical protocols indicate cord blood transplantation. Clinical trials aimed at hastening engraftment with the use of multiple cord-blood transplants, ex vivo expansion of cord-blood stem cells, and co-transplantation of CD34+ cells from a haploidentical donor are ongoing. It is realistic to anticipate that the use of cord-blood transplantation in adult and pediatric patients will continue to increase in the coming years.

### Allogeneic Hematopoietic Stem Cell Transplantation for Hematologic Malignancies

The outcome of unrelated donor hematopoietic stem cell transplantation has greatly improved over the last two decades as a result of a better understanding of the HLA barrier and advances in patient peri-transplant supportive care. Consequently, the indications for unrelated donor hematopoietic cell transplantation have broadened. Today, allogeneic hematopoietic stem cell transplantation is a therapeutic option, or even the treatment of choice in several high-risk hematologic malignancies.

### Acute Myeloid Leukemia (AML)

The optimal use of allogeneic hematopoietic stem cell transplantation for patients with AML has been debated for the last two decades. For patients in first remission, some studies indicate allogeneic transplantation to be associated with improved survival. The outcome of patients beyond first remission, in relapse or with primary induction failure, is poorer than for patients in first remission. However, studies show that allogeneic transplantation is a useful salvage therapy for these patients.

### Acute Lymphocytic Leukemia (ALL)

Clinical trials indicate superiority of allogeneic hematopoietic cell transplantation over autologous transplantation or chemotherapy in adults with high-risk ALL (Oh, H., et al. Bone Marrow Transplant, 1998; 22:253-7) and Philadelphia chromosome positive ALL (Dombret, H., et al. Blood; 2002, 100: 2357-66).

Cure is unlikely with conventional therapy in patients with ALL experiencing first relapse, and improved survival has been demonstrated with hematopoietic transplantation. When HLA-matched siblings are unavailable, unrelated donors may be used, resulting in improved disease free survival.

### Chronic Myeloid Leukemia (CML)

Efficacy of allogeneic stem cell transplantation has been widely validated; Imatinib mesylate is considered first line therapy in many centers for patients who lack an HLA-identical sibling donor. Outcomes of transplantation from unrelated donors are inferior to those of HLA identical siblings. Cord blood transplantation in CML has been described in several clinical series.

### Lymphoma

Patients with high-risk lymphomas have been managed over the past 2 decades with high-dose chemotherapy and autologous transplantation, however, the possibility of a graft vs. lymphoma effect has made allotransplantation for lymphoma more attractive. There are some indications of improved survival with this approach. Studies have shown that allogeneic stem cell transplantation may benefit some patients depending on donor availability, disease remission and performance status.

### Myelodysplastic Syndrome (MDS)

Numerous therapeutic strategies have been investigated for MDS. To date, stem cell transplantation is the only treatment with curative potential for these patients. However, as MDS often shows a protracted course and many patients are in their 60's or older, myeloablative stem cell transplantation is appropriate only for some.

### Cord Blood Transfusion for Hematologic Malignancies

Cord blood is being used increasingly as an alternative hematopoietic stem cell source for patients with hematologic malignancies. To date, over 6000 cord blood transplant procedures in children and adults have been performed worldwide.

The higher primary graft failure rates and delayed donor myeloid recovery in cord blood recipients are due to the low graft hematopoietic stem cell dose, which includes up to 10-fold fewer nucleated and CD34+ cells compared with marrow or peripheral blood donor grafts. Cord blood graft variables, that have predictive value for time-to-donor myeloid engraftment, include cryopreserved and re-infused total nucleated graft cell content, CD34 content and infused colony forming units (CFU).

Donor myeloid engraftment is delayed compared to conventional allogeneic grafts, and ranges from 22 to 30 days. The probability of engraftment ranges from 65% to 88%. Acute GvHD grades II-IV has ranged from 35% to 40%, despite most utilized grafts disparate at two or more HLA loci. Event-free survival falls in a broad range (22%-62%). High peri-transplant mortality is attributed in part to delayed donor myeloid recovery. Transplant outcomes for patients allografted with related cord blood have been similar or slightly better, compared to unrelated cord blood grafts.

Data regarding cord blood transplantation in adults are more limited, and despite the high-risk profile of most studies, two recent studies concluded that unrelated cord blood is an acceptable alternative source of hematopoietic stem cells for adults with acute leukemia who lack an HLA-matched marrow donor. There were no differences in the rate of recurrence of leukemia. Among cord blood recipients, outcomes were similar between grafts with 1 or 2 HLA mismatches.

To overcome the limitation of the low numbers of stem and progenitor cells, ex vivo expansion of cord blood stem cells and progenitors with a cocktail of growth factors has been attempted. Clinical trials conducted with ex vivo expanded cells in the presence of combinations of early and late acting cytokines have not proven efficacious, since the expanded cells supported by these combinations of cytokines contain mainly late progenitors and differentiated cells. It has been shown that a combination of early- and late-acting cytokines, including SCF, thrombopoietin (TPO), G-CSF and IL-3, resulted in only a marginal-fold expansion of late (CD34) and early (CD34CD38) progenitor cells, perhaps due the fact that the late-acting cytokines drive the cultures mainly toward accelerated differentiation. However, culturing the cord blood cells with only early-acting cytokines (SCF, TPO, IL-6 and FLT-3 ligand) resulted in better and prolonged expansion of both late and early progenitors, which are important for short-term early trilineage engraftment.

The present inventors have previously demonstrated that ex-vivo culture with a polyamine copper chelator increased the long-term expansion and engraftment potential of cord blood-derived progenitor cells (see, for example, U.S. Pat. Nos. 6,962,698; 6,887,704; 7,169,605 and 7,312,078). During the culture period, the copper chelator inhibited the onset of cytokine-driven differentiation of early progenitor cells, resulting in a robust accumulation of CD34+CD38− and CD34+Lin- cells, with no effect on proliferation and differentiation of more mature committed cells [CD34+Lin− and CFU culture (CFUc)]. Three-week treatment of cord blood cultures supplemented with early acting cytokines with the polyamine chelator TEPA results in prolonged and extensive ex vivo expansion of CD34+ cells and higher percentages of early progenitors (CD34+CD38−, CD34+CD38−Lin-). In long-term cultures TNC, CFU-C and CD34+ cell expansion persists. Engraftment potential of the TEPA treated cultures in NOD/SCID mice was increased as compared to untreated cultures. In addition, the percentage of engrafted human progenitors as well as that of myeloid and lymphoid cells was significantly higher in mice transplanted with TEPA-expanded cells in comparison with mice transplanted with non-expanded cells. These results strongly suggest that copper chelators support the self-renewal division cycle without compromising differentiation capacity of hematopoietic stem cells.

Peled et al (Cytotherapy, 2004, 6:344-55) have adapted methods for ex-vivo culture expansion of cord-blood derived hematopoietic progenitor cells with a polyamine copper chelator to comply with standards for clinical practice, and have demonstrated clinical scale expansion of hematopoietic progenitor cells ex-vivo according to the revised method, and that transplantation of the ex-vivo expanded cells to mice results in effective long-term engraftment. Using cord blood stem cells expanded ex-vivo with a copper chelator prior to infusion in combination with unmanipulated cord blood, the inventors have completed a phase I/II study of 10 subjects with acute leukemia and lymphoma receiving high-dose chemotherapy.

However, Peled et al. did not provide a clinically verifiable protocol for selection and exclusion of hematopoietic progenitor populations, following ex-vivo expansion, for effective transplantation into recipients.

Thus, there is a need for clinically verified protocols enabling identification of ex-vivo expanded populations of hematopoietic cells suitable for transplantation into recipients in need thereof, in the clinical setting.

### SUMMARY OF THE INVENTION

According to an aspect of some embodiments of the present invention there is provided a method of selecting a population of ex-vivo expanded hematopoietic stem cells suitable for transplantation, the method comprising: (a) determining prior to administration in a candidate population of expanded hematopoietic cells at least one of the following parameters: (i) proportion of CD34+ cells in the population; (ii) fold expansion of total cells in the population; (iii) viability of the cells in the population; and (iv) total number of viable cells; and (b) selecting or excluding the candidate population according to predetermined values of at least one of the parameters, thereby selecting a population of ex-vivo expanded hematopoietic stem cells suitable for transplantation. Also provided is an expanded hematopoietic stem cell population selected suitable for transplantation according to the claimed method.

According to an aspect of some embodiments of the present invention there is provided a method of treating a hematological disease or condition in a subject in need thereof, the method comprising administering to a subject in need thereof a population of expanded hematopoietic stem cells selected suitable for transplantation by (a) determining prior to administration in a candidate population of expanded hematopoietic cells at least one of the following parameters: (i) proportion of CD34+ cells in the population; (ii) fold expansion of total cells in the population; (iii) viability of the cells in the population; and (iv) total number of viable cells; and (b) selecting or excluding the candidate population according to predetermined values of at least one of the parameters, thereby treating and/or preventing the hematological disease in the subject.

According to some embodiments of the present invention the proportion of the CD34+ cells is about 3 to about 10 percent of total cells and optionally about 3 to about 5 percent of total cells.

According to yet further embodiments of the present invention the proportion of the CD34+ cells is about 4 percent of total cells.

According to some embodiments of the present invention the fold expansion of total cells of the population is about 10 to about 70 times, and optionally about 25 to about 60 times.

According to yet further embodiments of the present invention the fold expansion of total cells of the population is about 50 times.

According to some embodiments of the present invention the viability of total cells in the population is about 65 percent to about 95 percent at 21 days culture, optionally about 75 percent to about 90 percent at 21 days culture.

According to yet further embodiments of the present invention the viability of total cells in the population is about 85 percent at 21 days culture.

According to some embodiments of the present invention the total number of viable cells of the expanded population is about 15×10⁶ to about 60×10⁶ viable cells, optionally about 20×10⁶ to about 30×10⁶ viable cells.

According to yet further embodiments of the present invention the total number of viable cells of the expanded population is about 23×10⁶ viable cells.

According to some embodiments of the present invention the selecting is determined according to the values of at least two of the parameters and optionally at least three of the parameters.

According to yet further embodiments of the present invention the selecting is determined according to the values of all four of the parameters.

According to still further embodiments of the present invention the selecting is determined according to the following values of the parameters: (i) the proportion of the CD34+ cells being about 4 percent of total cells; (ii) the fold expansion of total cells of the population is about 50 times; (iii) the viability of total cells in the population is about 85 percent at 21 days culture; and (iv) the total number of viable cells of the expanded population is about 23×10⁶ viable cells. Also provided is an expanded hematopoietic stem cell population selected suitable for transplantation according to the claimed method.

According to some embodiments of the present invention the hematopoietic stem cells are expanded by propagation ex-vivo by culturing hematopoietic cells in the presence of cytokines and a copper chelator.

According to some embodiments of the present invention the cytokines are early acting cytokines, which may be selected from the group consisting of: stem cell factor, FLT3 ligand, interleukin-1, interleukin-2, interleukin-3, interleukin-6, interleukin-10, interleukin-12, tumor necrosis factor-□ and thrombopoietin.

According to yet further embodiments of the present invention the cytokines are stem cell factor, thrombopoietin, interleukin-6 and FLT3 ligand.

According to some embodiments of the present invention the cytokines are provided at a concentration of 50 ng/ml.

According to some embodiments of the present invention the copper chelator is tetrethylenepentamine (TEPA).

According to some embodiments of the present invention the tetraethylenepentamine is provided at a concentration of 5 μM.

According to some embodiments of the present invention the expanded hematopoietic stem cells have been cultured for 21 days.

According to some embodiments of the present invention the expanded hematopoietic stem cell population comprises at least 1×10⁵ cells prior to expansion.

According to some embodiments of the present invention the hematopoietic cell population is selected from a source consisting of umbilical cord blood, peripheral blood and bone marrow.

According to yet further embodiments of the present invention the hematopoietic cell population is cord blood.

According to some embodiments of the present invention the cord blood is thawed frozen cord blood.

According to some embodiments of the present invention the hematopoietic cell population has been enriched for hematopoietic stem cells prior to expansion.

According to yet further embodiments of the present invention the hematopoietic cell population has been enriched for CD34+ or CD133+ cells prior to expansion.

According to some embodiments of the present invention the hematological disease is selected from the group consisting of acute myelocytic leukemia (AML), acute lymphocytic leukemia (ALL), chronic lymphocytic leukemia (CLL), chronic myelocytic leukemia (CLL), Hodgkins lymphoma(HL), non-Hodgkins lymphoma (NHL) and myelodysplastic syndrome (MDS).

According to further embodiments of the present invention, the method of treating the hematological disease further comprises mixing the expanded hematopoietic cells with unexpanded hematopoietic cells prior to the administering the cells to the subject. The expanded and the unexpanded hematopoietic cells can be obtained from the same cord blood unit or units.

According to some embodiments of the present invention the unexpanded hematopoietic cells are administered prior to the expanded hematopoietic stem cell population.

According to some embodiments of the present invention the subject is treated with immunosuppressive treatment prior to the administration of the hematopoietic cells.

According to yet further embodiments of the present invention the subject is treated with immunosuppressive treatment following the administration of the hematopoietic cells.

According to some embodiments of the present invention the expanded hematopoietic cells are co-administered in conjunction with an additional treatment for hematological disease. The additional treatment may be selected from the group consisting of immunosuppressive treatment, chemotherapy and radio-therapy.

According to an aspect of some embodiments of the present invention there is provided an article of manufacture comprising a packaging material and a selected ex-vivo expanded hematopoietic cell population, the hematopoietic cell population selected suitable for transplantation by (a) determining prior to administration in a candidate population of expanded hematopoietic cells at least one of the following parameters: (i) proportion of CD34+ cells in the population; (ii) fold expansion of total cells in the population; (iii) viability of the cells in the population; and (iv) total number of viable cells; and (b) selecting or excluding the candidate population according to predetermined values of at least one of the parameters; and wherein the packaging material comprises a label or package insert indicating that the hematopoietic cell population is for treating a hematological disease or condition in a subject in need thereof.

According to some embodiments of the present invention the selecting is according to the following values of the parameters: (i) the proportion of the CD34+ cells being about 4 percent of total cells; (ii) the fold expansion of total cells of the population is about 50 times; (iii) the viability of total cells in the population is about 85 percent at 21 days culture; and (iv) the total number of viable cells of the expanded population is about 23×10⁶ viable cells.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF EMBODIMENTS OF THE INVENTION

The present invention relates to a method of selecting populations of hematopoietic cells for transplantation to subjects in need thereof. Specifically, ex-vivo expanded hematopoietic cell populations are selected according to predetermined criteria, to produce populations of expanded hematopoietic cells with a high probability of engraftment, for treatment of blood disorders, traumatic injuries and cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Allogeneic hematopoietic stem cell transplantation is a life-saving procedure for patients with hematologic disorders; yet wide application of this procedure is limited by the availability of suitably HLA-matched donors. Only 30% of patients who could benefit from this procedure have an HLA-matched sibling. Unrelated matched donors may be identified for approximately 75% of Caucasian patients through worldwide registries, but the lengthy search for a matched donor may critically delay transplantation. In addition, far fewer patients of racial minorities find suitable HLA-matched donors. Umbilical cord blood has been increasingly used as an alternative source of stem cells; however, its use in adults and adolescent patients is limited due to an insufficient cell dose required for satisfactory hematopoietic reconstitution.

Accordingly, cord blood grafts are routinely and more successfully used in the pediatric transplantation setting (only 20% of cord blood units in banks could suffice for a 75 kg patient according to the recommended threshold cell dose). Thus, adults receiving cord blood transplantation are at high risk of early mortality (as high as 40-60% day 100 mortality) and infection due to delayed engraftment and higher rates of engraftment failure.

Grafts with a higher number of nucleated cells and higher numbers of CD34+ cells result in higher probabilities of survival. Strategies to improve outcomes have included the use of two units for transplantation and the co-transplantation of CD34 or CD133-selected peripheral blood stem cells from a third party donor and one unrelated donor cord blood unit.

To address the problem of low progenitor cell dose, ex vivo expansion of cord blood-derived cells has been suggested. As yet, it is unclear whether expansion technology is able to provide a reliable, reproducible increase in the number of progenitor cells available from a single unit of cord blood, resulting in superior rates of engraftment and overall survival in adult patients. A significant challenge to presently available methods for graft production is the ability to generate an expanded population of committed hematopoietic progenitor cells without compromising the numbers of less differentiated progenitor cells (CD34+CD38− or CD34+Lin- cells). Culturing cord-blood derived hematopoietic stem and/or progenitor cells ex-vivo with early-acting cytokines, in the presence of a polyamine copper chelator results in significant inhibition of differentiation and expansion of the undifferentiated hematopoietic stem/progenitor population. Recent studies have shown that the ex-vivo expansion of desired hematopoietic cells in the presence of cytokines and copper chelators can be adapted to clinically acceptable standards for transplantation into a recipient in need thereof (Peled et al, Cytotherapy 2004; 6:344), and that patients transplanted with expanded cord blood cells demonstrated improved 100 day survival, no acute toxicity, no graft failures or early deaths, and no secondary engraftment failure (di Lima et al, Bone Marrow Transplant, 2008 Jan. 21, Epub).

The present invention provides criteria for selection of populations of expanded hematopoietic cells with a high probability of effective engraftment, the criteria including proportion of CD34+ cells, fold expansion of CD34+ cells, viability of the cells in the population and total number of viable cells. The selected populations can be used for transplantation in the clinical setting, for treatment of blood disorders, traumatic injuries and cancer.

Thus, according to an embodiment of the present invention, there is provided a method for selecting ex-vivo expanded hematopoietic stem and/or progenitor cell populations suitable for transplantation, the method comprising determining prior to administration in a candidate population of expanded hematopoietic cells at least one parameter selected from: proportion of CD34+ cells in said population; fold expansion of total cells in said population; viability of total cells in said population; and total number of viable cells in said expanded cell population; and selecting or excluding the candidate population according to predetermined values of at least one of said parameters, thereby selecting a population of ex-vivo expanded hematopoietic stem cells suitable for transplantation.

As used herein the term “ex-vivo” refers to a process in which cells are removed from a living organism and are propagated outside the organism (e.g., in a test tube, in a cell culture bag, etc).

As used herein, the term “in-vitro” refers to a process in which cells originating from a cell line or lines (such as embryonic cell lines, etc.) maintained in the laboratory, are manipulated outside of an organism. Such cell lines are often immortalized cells.

As used herein the phrase “population of cells” refers to a homogeneous or heterogeneous isolated population of cells which comprise cell populations potentially suitable for transplantation. In one embodiment of the present invention, at least a portion of the population of cells of this aspect of the present invention expresses CD34 and/or CD133 on the cell-surface.

As used herein, the phrase “stem cells” refers both to the earliest renewable cell population responsible for generating cell mass in a tissue or body and the very early progenitor cells, which are somewhat more differentiated, yet are not committed and can readily revert to become a part of the earliest renewable cell population. Hematopoietic stem cells are stem cells that can regenerate the cellular components of the blood, such as erythrocytes, leukocytes, platelets, etc.

As used herein, the phrases “non-stem”, “non-progenitor” and “committed cells” refer to cells at various stages of differentiation, which generally no longer retain the ability to revert to become a part of a renewable cell population. Methods of ex-vivo culturing stem, progenitor, and non-stem, non-progenitor committed cells are well known in the art of cell culturing. To this effect, see for example, the text book “Culture of Animal Cells—A Manual of Basic Technique” by Freshney, Wiley-Liss, N. Y. (1994), Third Edition, the teachings of which are hereby incorporated by reference.

As used herein, the term “viability” refers to the distinction between living and non-living cells. Cell viability may be judged by morphological changes or by changes in membrane permeability and/or physiological state inferred from the exclusion of certain dyes or the uptake and retention of others. Cell viability assays are well known in the art, including, but not limited to trypan blue or propidium iodide exclusion and rhodamine metabolic stain (Coder, D., Current Protocols in Cytometry, 1997, John Wiley and Sons, Inc., Unit 9.2, 9.2.1-9.2.14).

As used herein, the term “fold increase” refers to the portion of indicated cells present before expansion in culture as compared to the same cells counted following expansion for a given period. Doubling of the indicated cells, for example, is a 100% or 1.0 times fold increase. Fold increase can relate to all cells in a population (such as total nucleated cells), or any sub-population with a population of cells (for example, CD34+ cells).

As used herein, the term “expansion”, “expanded cells” or “expanded cell population” refers to cells that have been cultured under conditions resulting in increased numbers of the cells or cell populations, compared to numbers prior to culturing, or at another prior time point. Expansion may relate to all of the cells in a population, or to specific sub-populations within a cultured cell population. Expansion can be expressed as “fold increase”, “percentage increase”, etc.

As used herein, the term “about” refers to values within the range of 5% greater to 5% less than the indicated value. Thus, for example, “about 50 times (50×) fold increase” refers to a fold increase in the range of 47.5 times (47.5×) to 52.5 times (52.5×).

According to one embodiment of the invention as claimed, the hematopoietic stem cells are expanded by propagation ex-vivo by culturing hematopoietic cells with conditions for cell proliferation in the presence of cytokines and a copper chelator. Culturing the ex-vivo grown cells with the conditions for cell proliferation include providing the cells with nutrients.

Final concentrations of the copper chelator may be, depending on the specific application, in the micromolar or millimolar ranges. For example, within about 0.1 μM to about 100 mM, preferably within about 4 μM to about 50 mM, more preferably within about 5 μM to about 40 mM.

According to some embodiments of the invention the chelator is a polyamine chelating agent, such as, but not limited to ethylendiamine, diethylenetriamine, triethylenetetramine, triethylenediamine, tetraethylenepentamine, aminoethylethanolamine, aminoethylpiperazine, pentaethylenehexamine, triethylenetetramine-hydrochloride, tetraethylenepentamine-hydrochloride, pentaethylenehexamine-hydrochloride, tetraethylpentamine, captopril, penicilamine, N,N′-bis(3-aminopropyl)-1,3-propanediamine, N,N,Bis (2 animoethyl) 1,3 propane diamine, 1,7-dioxa-4,10-diazacyclododecane, 1,4,8,11-tetraaza cyclotetradecane-5,7-dione, 1,4,7-triazacyclononane trihydrochloride, 1-oxa-4,7,10-triazacyclododecane, 1,4,8,12-tetraaza cyclopentadecane or 1,4,7,10-tetraaza cyclododecane. In one embodiment the copper chelator is tetraethylenepentamine.

According to another embodiment of the invention the cytokines are early acting cytokines, such as, but not limited to, stem cell factor, FLT3 ligand, interleukin-1, interleukin-2, interleukin-3, interleukin-6, interleukin-10, interleukin-12, tumor necrosis factor-□ and thrombopoietin. In one embodiment the cytokines are a combination of cytokines including stem cell factor, FLT3 ligand, interleukin-6 and thrombopoietin. Additionally and optionally, late acting cytokines, such as, but not limited to, granulocyte colony stimulating factor, granulocyte/macrophage colony stimulating factor and erythropoietin can be added. Final concentrations of the cytokines may be, depending on the specific application, in the microgram per milliliter to about nanogram per milliliter range. For example, concentrations of cytokines can be within about 0.1 ng/ml to about 100 μg/ml, optionally within about 1 ng/milliliter to 20 μg/ml, optionally within about 5 ng/ml to about 1 μg/ml. In one embodiment cytokine concentration is 50 ng/ml per cytokine.

It is appreciated that culturing includes provision of nutrients, a suitable growth medium and conditions suitable for cell growth. Suitable culture media capable of supporting cells include MEM-alpha, HEM, DMEM, RPMI, F-12, and the like. If required, the medium can contain supplements required for cellular metabolism such as glutamine and other amino acids, vitamins, minerals and useful proteins such as transferrin, serum (fetal calf, horse, bovine) and the like. The medium may also contain antibiotics to prevent contamination with yeast, bacteria, and fungi, such as penicillin, streptomycin, gentamicin, and the like. When culturing the cells, conditions should be close to physiological conditions (preferably, a pH of about 6 to about 8, and a temperature of about 30° C. to about 40° C.).

In one embodiment, cells are cultured in a medium comprising minimum essential medium-alpha (MEM-α), 10% fetal calf serum (FCS Hyclone, Logan, Utah, USA), cytokines stem cell factor, FLT3 ligand, interleukin-6 and thrombopoietin, and tetraethylenepentamine as indicated.

Hematopoietic cells can be grown in culture for short term (days or few weeks) or long term (weeks to months) duration. In order to maintain adequate vitality of the cells for transplantation, while at the same time affording opportunity for significant expansion of hematopoietic stem cell populations, cells can be cultured for 2-5 weeks. In one embodiment, the cells are expanded by culturing for 21 days (3 weeks).

Many vessels suitable for culturing hematopoietic cell populations for expansion are known in the art, such as cell culture bags, flasks, bioreactors, and the like, of various capacity. In one embodiment, the cells are cultured in 72 ml VueLife Teflon PEP cell culture bags (American Fluoroseal Co.).

When cord blood is the source of cells for expansion, it is important to ensure adequate numbers of cells for expansion, so as to provide the potential of producing a population of expanded hematopoietic stem cells large enough for successful engraftment. Thus, according to one embodiment, cord blood units selected for expansion comprise at least at least 20×10⁵/kg body weight cells, optionally at least 10×10⁵/kg body weight cells and optionally at least 1×10⁵/kg body weight cells prior to expansion. Cord blood units having fewer than 1×10⁵/kg body weight cells are excluded from consideration.

The population of cells of the present invention may be from an autologous or non-autologous donor (allogeneic or xenogeneic).

According to one embodiment of the claimed invention, expanded hematopoietic stem cell populations are selected or excluded according to the proportion of CD34+ cells in the population, wherein populations having fewer than at least 10% CD34+ cells following ex-vivo expansion are excluded from suitability for transplantation. In another embodiment, expanded hematopoietic stem cell populations having fewer than 9% CD34+ cells, alternatively having fewer than 8% CD34+ cells, alternatively having fewer than 7% CD34+ cells, alternatively having fewer than 6% CD34+cells, alternatively having fewer than 5% CD34+ cells, alternatively having fewer than 4% CD34+ cells and alternatively having fewer than 3% CD34+ cells are excluded from suitability for transplantation. In yet another embodiment, ex-vivo expanded hematopoietic stem cell populations selected for transplantation have at least 4% CD34+ cells.

According to one embodiment of the claimed invention, expanded hematopoietic stem cell populations are selected or excluded according to the fold expansion of total nucleated cells in the population, wherein populations having less than at least 10 times (10×) fold increase of total nucleated cells following ex-vivo expansion are excluded from suitability for transplantation. In another embodiment, expanded hematopoietic stem cell populations having less than 20 times (20×) fold increase of total nucleated cells, alternatively having less than at least 30 times (30×) fold increase of total nucleated cells, alternatively having less than at least 40 times (40×) fold increase of total nucleated cells, alternatively having less than at least 50 times (50×) fold increase of total nucleated cells, alternatively having less than at least 60 times (60×) fold increase of total nucleated cells are excluded from suitability for transplantation. In yet another embodiment, ex-vivo expanded hematopoietic stem cell populations selected for transplantation have at least 50 times (50×) or greater fold increase of total nucleated cells.

According to a further embodiment of the claimed invention, expanded hematopoietic stem cell populations are selected or excluded according to the proportion of viable cells, from total cells, in the population, wherein populations having fewer than at least 90% viable cells following ex-vivo expansion are excluded from suitability for transplantation. In another embodiment, expanded hematopoietic stem cell populations having fewer than 85% viable cells, alternatively having fewer than 80% viable cells, alternatively having fewer than 75% viable cells, alternatively having fewer than 70% viable cells, alternatively having fewer than 65% viable cells, alternatively having fewer than 60% viable cells are excluded from suitability for transplantation. In yet another embodiment, ex-vivo expanded hematopoietic stem cell populations selected for transplantation have at least 70% or greater viable cells from the total number of expanded cells.

In another embodiment, ex-vivo expanded cell populations are selected or excluded according to the absolute numbers of CD34+ cells available for infusion, determined according to a combination of the parameters of the proportion of CD34+ cells in said population and the total number of viable cells in the population. In yet another embodiment, ex-vivo expanded cell populations are selected or excluded according to the numbers of CD34+ cells available for infusion, determined according to a combination of the parameters of the number of CD133+ cells seeded in culture and the fold expansion of total nucleated cells in the expanded population.

According to yet a further embodiment of the claimed invention, expanded hematopoietic stem cell populations are selected or excluded according to the total number of viable cells in the population, wherein populations having fewer than at least 60×10⁶ viable cells following ex-vivo expansion are excluded from suitability for transplantation. In another embodiment, expanded hematopoietic stem cell populations having fewer than 50×10⁶ viable cells, alternatively having fewer than 40×10⁶ viable cells, alternatively having fewer than 30×10⁶ viable cells, alternatively having fewer than 25×10⁶ viable cells, alternatively having fewer than 20×10⁶ viable cells, alternatively having fewer than 15×10⁶ viable cells are excluded from suitability for transplantation. In yet another embodiment, ex-vivo expanded hematopoietic stem cell populations selected for transplantation have at least 23×10⁶ or greater viable cells from the total number of expanded cells.

According to the present invention, expanded cell populations may be selected or excluded according to each of the parameters selected from the proportion of CD34+ cells in said population; the fold expansion of total cells in said population; the viability of total cells in said population; and total number of viable cells in said expanded cell population, or may alternatively be selected or excluded according to a combination of parameters. Thus, in yet another embodiment of the present invention, ex-vivo expanded cell populations may be selected or excluded according to at least two, optionally at least three, or optionally according to all four of the parameters including the proportion of CD34+ cells in said population; the fold expansion of total cells in said population; the viability of total cells in said population; and total number of viable cells in said expanded cell, in any combination thereof. By way of illustration, ex-vivo expanded hematopoietic stem cell populations selected suitable for transplantation can include populations having at least 4% CD34+ cells and at least 50 times (50×) or greater fold increase of total nucleated cells, or populations having at least 50 times (50×) or greater fold increase of total nucleated cells and having at least 23×10⁶ or greater viable cells from the total number of expanded cells or populations having at least 4% CD34+ cells and at least 50 times (50×) or greater fold increase of total nucleated cells and at least 23×10⁶ or greater viable cells from the total number of expanded cells. In one embodiment, ex-vivo expanded cell populations are selected or excluded according to all four of the parameters, ex-vivo expanded hematopoietic stem cell populations being selected for transplantation having at least 4% CD34+ cells and at least 50 times (50×) or greater fold increase of total nucleated cells and at least 70% or greater viable cells from the total number of expanded cells and at least 23×10⁶ or greater viable cells from the total number of expanded cells.

In one embodiment, the cells for transplantation are stem and/or progenitor cells, and the source of the stem cell population is an unfractionated mononuclear cell preparation, not having been enriched for CD34+, CD133+ or other hematopoietic stem cells. In another embodiment, the stem cells are identified by stem cell markers such as CD34+, CD34+/CD38−, CD133+, CD34+/Lin-, and other stem cell markers known in the art. In yet another embodiment, the source of the stem cell population are stem cells having been enriched for hematopoietic stem cells by selection according to stem cell markers. Selection is usually by FACS, or immunomagnetic separation, but can also be by nucleic acid methods such as PCR (see Materials and Experimental Methods hereinbelow). Embryonic stem cells and methods of their retrieval are well known in the art and are described, for example, in Trounson A O (Reprod Fertil Dev (2001) 13: 523), Roach M L (Methods Mol Biol (2002) 185: 1), and Smith A G (Annu Rev Cell Dev Biol (2001) 17:435). Adult stem cells are stem cells, which are derived from tissues of adults and are also well known in the art. Methods of isolating or enriching for cord blood and adult stem cells are described in, for example, Miraglia, S. et al. (1997) Blood 90: 5013, Uchida, N. et al. (2000) Proc. Natl. Acad. Sci. USA 97: 14720, Simmons, P. J. et al. (1991) Blood 78: 55, Prockop DJ (Cytotherapy (2001) 3: 393), Bohmer R M (Fetal Diagn Ther (2002) 17: 83) and Rowley S D et al. (Bone Marrow Transplant (1998) 21: 1253), Stem Cell Biology Daniel R. Marshak (Editor) Richard L. Gardner (Editor), Publisher: Cold Spring Harbor Laboratory Press, (2001) and Hematopoietic Stem Cell Transplantation. Anthony D. Ho (Editor) Richard Champlin (Editor), Publisher: Marcel Dekker (2000).

For example, stem cells of the present invention may be derived from a source selected from the group consisting of hematopoietic cells, umbilical cord blood cells, and mobilized peripheral blood cells.

According to yet another exemplary embodiment of the invention, there is provided an expanded hematopoietic cell population or populations selected suitable for transplantation by the claimed methods. Such selected cell populations can be used for transfusion after selection, or preserved for future use. Methods of preservation of hematopoietic cell populations are well known in the art, such as cryopreservation, freeze-drying and the like (see, Watts et al, Cryopreservation and Freeze-Drying Protocols, in Methods in Molecular Biology, 2007; 368:237-259).

In one embodiment, the expanded cell population comprises ex-vivo expanded hematopoietic stem cell populations having at least 4% CD34+ cells and at least 50 times (50×) or greater fold increase of total nucleated cells and at least 70% or greater viable cells from the total number of expanded cells and at least 23×10⁶ or greater viable cells from the total number of expanded cells.

Hematopoietic stem cell populations selected according to the methods of the present invention can be used for transplantation into subjects in need thereof, for example, subjects suffering from a hematological disease or condition. Thus, according to a further aspect of the claimed invention there is provided a method of treating a hematological disease or condition in a subject in need thereof, the method comprising administering to a subject in need thereof a population of expanded hematopoietic stem cells selected suitable for transplantation by (a) determining prior to administration in a candidate population of expanded hematopoietic cells at least one of the following parameters: (i) proportion of CD34+ cells in said population; (ii) fold expansion of total cells in said population; (iii) viability of said cells in said population; and (iv) total number of viable cells; and (b) selecting or excluding said candidate population according to predetermined values of at least one of said parameters, thereby treating and/or preventing said hematological disease in said subject. In one embodiment, the expanded cell population comprises ex-vivo expanded hematopoietic stem cell populations having at least 4% CD34+ cells and at least 50 times (50×) or greater fold increase of total nucleated cells and at least 70% or greater viable cells from the total number of expanded cells and at least 23×10⁶ or greater viable cells from the total number of expanded cells.

The following summarizes some clinical applications which may be addressed according to the teachings of the present invention.

Hematopoietic cell transplantation: restoring hematopoiesis in recipients with completely ablated bone marrow, as well as in providing a supportive measure for shortening recipient bone marrow recovery following conventional radio- or chemo-therapies.

Recent reports have demonstrated the capability of transplanted or transfused stem cells to enhance regeneration in non-homologous tissue, other than that which the stem cells were derived. Thus, the selected cell populations can be used for tissue regeneration, regenerative medicine, reconstructive surgery, tissue engineering, regenerating new tissues and naturally healing diseased or injured organs.

Cells of selected populations can be genetically modified prior to, during or following expansion in culture. Gene transfer into fresh stem cells is highly inefficient. The ability to store and process a selected population of cells ex-vivo, and enhance their homing and engraftment potential would provide for an increased probability of the successful use of genetically modified cell transplantation for gene therapy. Methods of genetic manipulation of hematopoietic cells, such as the use of retroviral vectors, are well known in the art (see, for example, Larochelle et al, Semin. Hematol. 2004;41:257-71).

Hematological diseases or conditions that can be treated or prevented by transplantation of hematopoietic stem cell populations selected according to the present invention include, but are not limited to, leukemia, sickle-cell disease, myelodysplastic syndrome, neuroblastoma, lymphoma, Ewing's Sarcoma, Desmoplastic small round cell tumor, Hodgkin's disease, non-Hodgkin's lymphoma and multiple myeloma.

Candidates for receiving transplantation of selected hematopoietic stem cell populations may require conditioning or other care prior to, during or following the transplantation, such as, but not limited to, myeloablative or non-myeloablative doses of chemotherapy (cyclophosphamide, busulfan) and/or irradiation to help eradicate the patient's disease prior to the infusion and to suppress immune reactions, tacrolimus and methotrexate for Graft vs. Host Disease (GVHD) prophylaxis, antibiotics and blood products for supportive care.

Selected cell populations of the present invention can be provided per se, along with the culture medium containing same, isolated from the culture medium, and combined with a pharmaceutically acceptable carrier as well as with additional agents which may promote cell engraftment and/or organ function (e.g., immunosuppressing agents, antibiotics, growth factor). Hence, cell populations of the invention can be administered in a pharmaceutically acceptable carrier or diluent, such as sterile saline and aqueous buffer solutions. The use of such carriers and diluents is well known in the art.

Compositions of the present invention may, if desired, be presented in a pack or dispenser device, such as an FDA-approved kit, which may contain one or more unit dosage forms containing the active ingredient (e.g., cells). The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

The cells prepared according to the methods of the present invention can be administered to the subject per se, or in a pharmaceutical composition where it is mixed with suitable carriers or excipients.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient.

Techniques for formulation and administration of drugs may be found in the latest edition of “Remington's Pharmaceutical Sciences,” Mack Publishing Co., Easton, Pa., which is herein fully incorporated by reference.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g. expanded hematopoietic stem cells) effective to prevent, alleviate, or ameliorate symptoms of a disorder (e.g., leukemia) or prolong the survival of the subject being treated.

Determination of a therapeutically effective amount is well within the capability of those skilled in the art, especially in light of the detailed disclosure provided herein.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Depending on the severity and responsiveness of the condition to be treated, dosing can be of a single or a plurality of administrations. Commonly, however, subjects receive a single transplant, with multiple transplants being extremely rare. However, the amount of a composition to be administered will, of course, be dependent on the subject being treated, the severity of the affliction, the manner of administration, the judgment of the prescribing physician, etc.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Additional objects, advantages, and novel features of the present invention will become apparent to one ordinarily skilled in the art upon examination of the following examples, which are not intended to be limiting. Additionally, the various embodiments and aspects of the present invention as delineated hereinabove and as claimed in the claims section below finds experimental support in the following examples.

### EXAMPLES

Reference is now made to the following examples, which together with the above descriptions, illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Treatment of Hematological Malignancies with Selected Populations of Ex-Vivo Expanded Cord Blood

In order to determine the efficacy of imposing cell population selection criteria on success of cord blood-derived hematopoietic stem cell transplantation in the clinical setting, outcomes such as time to engraftment, percent engraftment, side effects and survival are monitored in subjects with hematological malignancies (AML, ALL, CML, MDS, HD or NHL) receiving transplantation of selected ex-vivo expanded cord blood HSC along with unexpanded cord blood cells as compared to similar subjects receiving transplantation of a single, unmanipulated cord blood unit.

### Materials and Methods

### Subject Population:

Subjects with hematologic malignancies for whom myeloablative stem cell transplantation is currently a recommended and potentially life saving treatment are included. Only adults and adolescent subjects 12 years of age and older who do not have an adequate alternative bone marrow donor are enrolled.

Criteria for subject's inclusion in the treatment group are:

A: Hematological diseases including:

1. Acute Myelocytic Leukemia (AML): Complete Remission 2 (CR2) or subsequent complete remission

Or CR1 with high-risk features including any of the following: stem cell or biphenotypic classification (AML-M0), erythroleukemia (AML-M6), acute megakaryocytic leukemia (AML-M7), high-risk cytogenetics, or failure to achieve CR after standard induction therapy or presentation with extramedullary disease;

Or relapse with <10% blasts in BM and no circulating blasts.

2. Acute Lymphocytic Leukemia (ALL): CR2 or subsequent CR

Or CR1 with high-risk features including: high-risk cytogenetics or with failure to achieve CR after standard induction therapy or elevated WBC at presentation (50,000 age <17 or 20,000 age 17) or presentation with extramedullary disease

Or relapse with <10% blasts in BM and no circulating blasts.

3. Chronic Myelocytic Leukemia (CML): in Chronic Phase 1 (CP1) and resistant or intolerant to Gleevec

Or in CP2 or subsequent CP;

Or in accelerated phase.

4. Hodgkin's Disease (HD): sensitive to last chemotherapy course and one of the following:
2nd relapse or
failure of autologous transplantation or
in first relapse and not eligible for autologous transplantation due to extensive bone marrow involvement.

5. Non-Hodgkins Lymphoma (NHL): sensitive to last chemotherapy course and one of the following:
2nd relapse or
failure of autologous transplantation or
in first relapse and not eligible for autologous transplantation due to extensive bone marrow involvement.

6. Myelodysplastic Syndrome (MDS) with intermediate 2—or high-risk IPSS score.

B: Age >12 and <55.

C: Availability of cord blood unit that meets the following requirements:

1. Matched at 4, 5, 6/6 HLA class I (HLA-A & HLA-B, low resolution) and II (HLA-DRB1, high resolution).

2. The cord blood unit undergoes volume reduction prior to cryopreservation and is preserved in two portions, of which the larger (or equal) portion contains a minimum of 1.0×10⁷ total nucleated cells/kg (pre-thaw).

3. Cord blood units are obtained from public cord blood banks where they are tested for infectious agents in compliance with the applicable local requirements and regulations.

D: Subjects' Performance score 70% by Karnofsky (age 13) or a Lansky Play-Performance scale of 70% (age <13).

E: Subject has sufficient physiologic reserves including:

1. Left ventricular ejection fraction_40%.

2. Pulmonary function test demonstrating a corrected CO₂ diffusion capacity 50% predicted.

3. Serum Creatinine <1.6 mg/dl.

4. Serum Bilirubin <2.0×upper limit of normal range.

5. SGPT (ALT)_{—}3.0×upper limit of normal range.

F: Subject has at least one back-up stem cell sources in case of engraftment failure:

1. Subject is willing to undergo bone marrow harvest or peripheral blood progenitor cell (PBPC) collection for use in case of engraftment failure (when clinically applicable).

2. Subject has a second cord blood unit as a possible back up.

3. Subject's haplo-identical family member has been identified and agreed (by signing a written informed consent) to donate hematopoietic stem cells in case of engraftment failure.

G. Subject or guardian signs a written informed consent after being aware of the nature of the subjects' disease and willingly consents to the treatment program after being informed of alternative treatments, potential risks, benefits, and discomforts.

Criteria for subject's exclusion from the treatment group are:

A. Less than twenty-one days have elapsed since the subject's last radiation or chemotherapy prior to conditioning (except Hydroxyurea).

B. HIV positive.

C. Pregnancy or lactation.

D. Uncontrolled bacterial, fungal or viral infection.

E. Subjects with signs and symptoms of active central nervous system (CNS) disease.

F. Availability of appropriate related and willing stem cell donor, who is HLA-matched at 5 or 6/6 antigens.

G. Prior allogeneic cell transplant.

H. Allergy to bovine or to any product, which may interfere with the treatment.

I. Enrolled in another clinical trial or received an investigational treatment during the last 30 days.

### Subject Screening

Upon identification of an acceptable cord blood unit, eligible subjects are screened from 3 to 5 weeks before transplantation. The subject or his/her legal guardian are thoroughly informed and sign the written informed consent. Screening activities include:

### Routine Baseline Evaluation:

Medical history including primary and concomitant disease and concomitant medications,
and prior radio- and chemotherapeutic courses.
Performance status: Karnofsky Scale (age >13) or Lansky Score (age 13).
Physical examination and vital signs (including height and weight).
Routine laboratory: CBC, blood chemistry, PT, PTT, urinalysis, serum betaHCG (females) (tests from last week prior to screening are acceptable).
Immunophenotyping—Lymphocyte subsets: CD3, CD4, CD8, CD19, CD16/56
Immunoglobulin levels (IgG, IgA, IgM, IgE)
Chest-X ray (tests from last month prior to screening are acceptable).

### Disease Status Evaluations:

BM (aspiration/biopsy, as clinically indicated) morphology
For Leukemia or MDS: Peripheral blood and BM (aspiration/biopsy, as clinically indicated) morphology: FACS assay, cytogenetics and molecular markers.
For NHL and HD CT abdomen, pelvis & chest (tests from last month prior to screening are acceptable).
CT sinuses (tests from last month prior to screening are acceptable).

### Cord Blood Match:

Subjects and cord blood unit ABO and Rh typing.
Subject HLA class I & II high resolution typing.
Peripheral blood baseline sample for chimerism laboratory.
Backup leukapheresis/BM harvest as applicable

### Serology Screen:

Serologic tests for HIV 1&2 Ab, HTLV 1&2 Ab, HbsAg, HbcAb, HCV Ab, HSV Ab, VZV Ab, EBV Ab, Toxoplasmosis Ab, CMV Ab, Dental Consultant (recommended, optional).

### Immunophenotyping—Lymphocyte Subsets: CD3, CD4, CD8, CD19, CD16/56

### Immunoglobulin Levels (IgG, IgA, IgM, IgE)

### Physiologic Reserves Assessment:

Pulmonary Function Test including Carbon Monoxide Diffusing Capacity (DLCO) (tests from last month prior to screening are acceptable).
12 lead ECG (test from last month prior to screening is acceptable).
Echocardiography or MUGA with left ventricular ejection fraction (LVEF) (test from last month prior to screening is acceptable).
For subjects with an autologous stem cell backup, harvesting of backup stem cells from subject (Leukopheresis or BM).

All abovementioned activities are completed prior to initiation of myeloablative preparatory regimen.

### Myeloablative Treatment

Subjects are myeloablated with one of three myeloablative regimens is selected, as follows, prior to cord blood transplantation:

### Regimen A:

Melphalan/Thiotepa/Fludarabine/Antithymocyte globulin (Mel/Thio/Flu/ATG):

### Day of Treatment

9 Hydration therapy
8 Melphalan 140 mg/m2, IV
7 Thiotepa 10 mg/kg, IV
6 Fludarabine 40 mg/m2, IV
5 Fludarabine 40 mg/m2, IV
4 Fludarabine 40 mg/m2, IV
3 Fludarabine 40 mg/m2, IV ATG 1.50 mg/kg, IV
2 ATG 1.25 mg/kg, IV
1 ATG 1.25 mg/kg, IV

### Regimen B:

Busulfan/Cyclophosphamide/ATG (Bu/Cy/ATG):

Dose adjustment of Busulfan based on pharmacokinetics, is recommended (Regimen B.1), or a fixed dose regimen (Regimen B.2) is used.

### Regimen B.1:

Bu/Cy/ATG with PK Dose Adjusted BU

### Day of Treatment

11 Hydration therapy
10 Busulfan 32 mg/m2, IV
9 Rest
8 Busulfan dose adjusted*by PK to AUC 6,000, IV (3-hours infusion)
7 Busulfan dose adjusted*by PK to AUC 6,000, IV (3-hours infusion)
6 Busulfan dose adjusted*by PK to AUC 6,000, IV (3-hours infusion)
5 Busulfan dose adjusted*by PK to AUC 6,000, IV (3-hours infusion)
4 Rest
3 Cyclophosphamide 60 mg/Kg, IV ATG 1.50 mg/kg, IV
2 Cyclophosphamide 60 mg/Kg, IV ATG 1.25 mg/kg, IV
1 ATG 1.25 mg/kg, IV

### Regimen B.2:

Fixed Dose Busulfan(Bu/Cy/ATG with Fixed Dose BU):

### Day of Treatment

9 Hydration therapy
8 Busulfan 130 mg/m², IV (3-hours infusion)
7 Busulfan 130 mg/m², IV (3-hours infusion)
6 Busulfan 130 mg/m², IV (3-hours infusion)
5 Busulfan 130 mg/m², IV (3-hours infusion)
4 Rest
3 Cyclophosphamide 60 mg/Kg, IV ATG 1. 50 mg/kg, IV
2 Cyclophosphamide 60 mg/Kg, IV ATG 1.25 mg/kg, IV
1 ATG 1.25 mg/kg, IV

### Regimen C:

### TBI/Cyclophosphamide/Fludarabine/ATG (TBI/Cy/Flu/ATG).

This regimen may be used in all subjects except subjects with HD who previously received an auto transplantation.

### Day, Agent and Route of Administration

10 Hydration therapy (2 L/m2/24 hours)
9 FTBI*150 cGy×2
8 FTBI*150 cGy×2
7 FTBI*150 cGy×2
6 FTBI*150 cGy×2
5 Cyclophosphamide 60 mg/kg/day, IV Fludarabine 30 mg/m2, IV
4 Cyclophosphamide 60 mg/kg/day, IV Fludarabine 30 mg/m2
3 Fludarabine 30 mg/m2 IV ATG 1.50 mg/kg/day, IV
2 Fludarabine 30 mg/m2 IV ATG 1.25 mg/kg/day, IV
1 ATG 1.25 mg/kg/day, IV

*Fractionated Total Body Irradiation (FTBI) doses may be divided once a day or 3×/day. Maximal total dose should not exceed 1200 cGy.

### GVHD Prophylaxis

One of the following two GvHD-prophylaxis regimens are initiated on day-1:

### Regimen A

### Tacrolimus/MMF:

Tacrolimus (FK506, prograf) 0.03 mg/kg/day IV or 0.10-0.15 mg/kg/day PO starting on day-2 until day 180.

Mycophenolate (MMF): The dose of MMF is 15 mg/Kg orally twice daily, with a maximum dose of 1 gram twice daily (i.e. a maximum total daily dose of 2 gram) starting on day-2 until day 100. Dose is adjusted for tablet size. Intravenous route is used if PO not tolerated (with the same dosing).

### Regimen B

### Cyclosporin/MMF:

Cyclosporine A (IV or PO) 1.5 mg/kg BID from day-2 until day 180. Mycophenolate (MMF): The dose of MMF is 15 mg/Kg orally twice daily, with a maximum dose of 1 gram twice daily (i.e. a maximum total daily dose of 2 gram) starting on day-2 until day 100. Dose is adjusted for tablet size. Intravenous route is used if PO not tolerated (with the same dosing).

### Baseline Evaluation Prior to Transplantation:

### Safety Assessment

Prior to transplantation the subject is evaluated as by physical examination, CBC, blood chemistry, urinalysis, vital signs (temperature, blood pressure, pulse, respiratory rate and saturation, and weight), cardiac status (One Lead Monitor: prior to transplantation, cardiac monitoring is initiated (1-lead) and continued until 24 hours after transplantation of the selected, expanded stem cell population).

### Cord Blood Units

Potential candidates for cord blood transfusion for whom a search yielded a matched cord blood unit are identified as screen candidates for the study. The cord blood units undergo volume reduction prior to cryopreservation and are cryopreserved in two portions. The larger (or equal) portion has at least 1×10⁷ total nucleated cells/kg subject's body weight. HLA type I & II matching of at least 4/6 loci is required (low resolution HLA-A & HLA-B, high resolution HLA-DRB1). Cord blood units are obtained from public banks where they are tested for infectious agents in compliance with the local applicable requirements and regulations.

### Ex-vivo Expansion of Cord Blood

Frozen cord blood units are stored in liquid nitrogen until use. Two fractions are separated, and the smaller or equal fraction thawed, washed in 10% w/v Dextran and 5% w/v human serum albumin (Aventis, Bridgewater N.J., USA). Cells are incubated with 0.15% w/v intravenous immunoglobulin (IvIg, Baxter, Deerfield Ill., USA) for 10 minutes at room temperature, centrifuged, and resuspended in PBS containing 0.4% sodium citrate, 1% human serum albumin and 0.2 mg/ml rHu-DNase (Genentech, San Francisco, Calif., USA). Cells are then labeled with CliniMACS CD 133+magnetic cell separation reagent (Miltenyi Biotech, Auburn, Calif., USA) and separated by CliniMACS according to manufacturers instructions. Following selection by CD133, cells are stained with trypan blue, counted, assayed for CFUc and immunophenotyped to determine purity. Purified CD133+ cells are then cultured in culture bags (American Fluoroseal Co., Gaithersburg Md., USA) at a concentration of 1×10⁴ cells per ml in minimum essential medium-alpha (MEM-α) and 10% fetal calf serum (FCS, Hyclone Logan Utah, USA) containing the following cytokines: stem cell factor, TPO, IL-6 and FLT-3 at final concentrations of 50 ng/ml (R&D Systems, Minneapolis Minn., USA) and 5 μM tetraethylenepentamine (TEPA) (Sigma, St Louis, Mo., USA). Expansion is performed in 72 ml VueLife Teflon PEP cell culture bags (American Fluoroseal Co) if the number of cells recovered following enrichment is equal to or less than 19×10⁴, and in a 270 ml VueLife Teflon PEP cell culture bag (American Fluoroseal Co) if the number of cells is between 20 and 70×10⁴.

The cultures are incubated for 3 weeks at 37° C. in a humidified atmosphere of 5% CO₂ in air. Cultures are topped up weekly with the same volume of fresh medium, FCS, growth factors and TEPA. At 3 weeks, cells are washed, and suspended in 100 ml PBS/EDTA/Human Serum Albumin infusion buffer. A sample of the expanded cells in infusion buffer is counted following vital stain (Trypan blue), assayed for CFUc and immunophenotyped for surface antigen analysis (CD34, CD38, CD133). Cells are administered to recipient as detailed herein. Aliquots are tested throughout the process for mycoplasma, endotoxin, sterility and Gram's staining.

### Thawing and Infusion of Unexpanded Cord Blood

Unit is washed to remove DMSO, or diluted to a final DMSO concentration of 2.5%. If the DMSO is washed away the frozen cord blood unit portion is placed in a sterilized zip lock bag. The bag is submerged in a 37° C. water bath and agitated until almost all ice crystals have dissolved. 10% Dextran-40 (9.5 ml) is slowly injected into the unit followed by 5% Human Serum Albumin (HSA) (9.5 ml). The suspension is transferred to a 150 ml transfer pack and diluted further with 40 ml of 10% Dextran-40 and then centrifuged. The supernatant is then discarded and the cord blood cell pellet is re-suspended in 50 ml of a solution containing 50% of the 10% Dextran-40 and 50% of the 5% HSA. The exact volume of the sample is measured and recorded and an aliquot removed to measure the total number of cells in the portion. An additional 500 μl of the sample is removed for the determination of the percentage of the CD34+ and CD133+ populations by labeling with fluorescent antibodies and detection by FACS analysis. The cord blood suspension is then transferred to the appropriate infusion bag for infusion via the subject's central venous catheter at a rate of 1-3 ml/min (no irradiation). If the subject develops chest tightness or other symptoms, a brief rest (1-2 minutes) is required before proceeding with the remainder of the infusion. Hydration (2.5-3.0 ml/kg/hr) is maintained for 12 hours after cord blood infusion is completed. Furosemide (0.5-1.0 mg/kg/dose) can be given if volume overload or decreased urine output occurs.

### Post-transplantation Follow-up and Safety Assessment Prior to Transplantation of Expanded, Selected HSC:

Safety assessment including: vital signs (temperature, blood pressure, pulse, respiratory rate and saturation) 15, 30, 60 minutes and at 2, 4 and 24 hours post transplantation, CBC, blood chemistry, urinalysis, Cardiac (One Lead Monitor) and adverse effects recording.

### Supportive Cytokine Therapy Initiation:

G-CSF (granulocyte colony-stimulating factor, filgrastim) is administered at a dose o 5 μg/kg/day, SC daily starting on day 0 until the ANC>2.5×10⁹/L.

### Transplantation of Selected, Expanded HSC Population

Quality Control tests are performed, including bioassays and safety tests, endotoxin and Gram stain tests. If the expanded selected HSC population does not meet any of the specifications for the safety tests (endotoxin content and Gram stain) it is destroyed. If, however, selected, expanded HSC population does not meet the specifications for the bioassays it is shipped to the clinical site, and can be used. The expanded, selected HSC bag is thoroughly but gently mixed by massaging it prior to infusion via the subject's central venous catheter with an in-line filter, at a rate of 1-3 ml/min. As needed, the rate of infusion is adjusted to a slower rate so that endotoxin infusion will not exceed 5 Eu/kg/hr, according to the levels of endotoxin in the specific expanded selected HSC population. If a large (or equal) volume of expanded selected HSC (>15 ml/kg) is to be infused, half the volume is infused, followed by a 30-minute rest period, and then infusion of the remainder of the volume. Hydration (2.5-3.0 ml/kg/hr) is maintained for 12 hours after the cord blood infusion is completed. Furosemide (0.5-1.0 mg/kg/dose) is given if volume overload or decreased urine output occurs. Safety assessment following transplantation includes vital signs (temperature, blood pressure, pulse, respiratory rate and saturation) 15, 30, 60 minutes and at 2, 4 and 24 hours post transplantation, CBC, blood chemistry, urinalysis, Cardiac (One Lead Monitor) and adverse effects recording.

### Follow Up

Follow up visits with standard assessment plus Tacrolimus/cyclosporine levels, immunological workup, adverse effects, CMV and GvHD assessment, bone marrow and peripheral blood evaluation (for chimerism, molecular markers and cytogenics), immunotyping, CT (lymphoma only) are carried out at predetermined days following final transplantation. Engraftment is evaluated at +35 days from bone marrow aspiration and peripheral blood samples. Graft rejection is defined by absence of donor hematopoiesis with evidence of host hematopoiesis on or before day +42.

### Statistics

Previous results (with 10 subjects) indicate a 20% 100 day mortality rate for patients receiving expanded HSC. Preliminary analysis of the combined records of the NCBP and the Center for International Blood and Marrow Transplant Research (CIBMTR) registries, totaling 431 cord blood transplant recipients aged 12 to 55 years with one of the indications (AML, ALL, HD, NHL), showed a mortality rate of 46% at 100 days. To be conservative we use 43% as the 100-day overall mortality in the historical control group in our statistical power calculations.

Accordingly, the statistical power for various levels of transplanted subjects 100-day mortality rates (20%, 25%, 27%, 30%) assuming a two-sided likelihood ratio test at the 5% level of significance correspond to odds ratios of 0.33, 0.44, 0.49 and 0.57 respectively compared to the mortality rate of 43% in the control group.

Engraftment failure monitoring is based on the calculation of the Bayesian posterior probability that the neutrophil engraftment failure rate exceeds 17% (approximately that experienced by control patients). Acute GvHD monitoring is based on the calculation of the Bayesian posterior probability that the aGvHD grade III-IV rate exceeds 19% (approximately that experienced by control patients). 100-day overall mortality monitoring will be calculated based on the Bayesian posterior probability that the 100-day overall mortality rate will exceed 0.43 (approximately that experienced by control patients).

Overall significance level is a one-tailed 2.5%, using logistic regression and a likelihood ratio test. This is equivalent to a two-tailed 5% significance level.

### Evaluation of Expanded Hematopoietic Cell Populations

Expansion parameters of progenitor/stem cell populations are estimated based on the pre-infusion counts in the unexpanded cord blood portions. Parameters assayed include:

### Cryopreserved Total Nucleated Cells:

cell dose/Kg recipient body weight

### Infused Cells:

Unexpanded cells and expanded populations: cell dose (total cells)/kg recipient body weight. Fold increase in expanded cells only.

### CD34+ Cells:

Unexpanded cell population only: absolute cell dose; cell dose/kg recipient body weight. Expanded and unexpanded portions: absolute cell dose; cell dose/kg recipient body weight. Expanded portion only: absolute cell dose; cell dose/kg recipient body weight; percent from total cells; fold expansion.

### CD133+ Cells:

Unexpanded cell population only: cell dose/kg recipient body weight. Expanded and unexpanded portions: absolute cell dose; cell dose/kg recipient body weight. Expanded cells only: absolute cell dose; cell dose/kg recipient body weight; percent from total cells; fold expansion.

### CD38− Cells:

Expanded cells only: absolute cell dose; cell dose/kg recipient body weight.

### CD38+ Cells:

Expanded cells only: absolute cell dose; cell dose/kg recipient body weight.

### CXCR4+ Cells:

Expanded cells only: absolute cell dose; cell dose/kg recipient body weight.

### CFU Cells:

Expanded cells only: cell dose/kg recipient body weight. Fold increase.

Using regression analyses, the parameters are then correlated with a clinical outcomes, as detailed herein (time to engraftment, percentage engraftment, GvHD, toxicity, 100 day, 180 day survival, disease free survival, neutrophil engraftment, platelet engraftment, infection). Univariate relationships are established first, and then for each clinical outcome a model is constructed based on the parameters of (i) proportion of CD34+ cells; (ii) fold expansion of total cells; (iii) percent viability of total cells at 21 days culture; and (iv) total number of viable cells.

### RESULTS

### Effect of Selection on Efficacy of Engraftment and Clinical Outcomes of Transplantation with Expanded Hematopoietic Cord Blood Stem Cell Populations in Hematological Disease.

Comparison of clinical outcomes (GvHD, toxicity, 100 day, 180 day survival, disease free survival, infection) and engraftment parameters (time to engraftment, percentage engraftment, neutrophil engraftment, platelet engraftment) in subjects suffering from hematological malignancies receiving both unexpanded and expanded cord blood populations, with those of subjects receiving only unexpanded cord blood, indicates the effect of added expanded HSC on efficacy of transplantation. Expanded cord blood populations selected according to proportion of CD34+ cells, fold expansion, viability of total cells and total number of viable cells, and characterized by the proportion of the CD34+ cells being about 4 percent of total cells; the fold expansion of total cells of the population being about 50 times; viability of total cells in the population being about 85 percent at 21 days culture; and total number of viable cells of the expanded population being 23×10⁶ viable cells are expected to produce superior clinical outcome and improved engraftments parameters.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### LIST OF REFERENCES

Coder, D., Current Protocols in Cytometry, 1997, John Wiley and Sons, Inc., Unit 9.2, 9.2.1-9.2.14
di Lima et al, Bone Marrow Transplant, 2008, January 21, Epub.
Dombret, H., et al. Blood; 2002, 100: 2357-66
Larochelle et al, Semin. Hematol. 2004; 41:257-71
Oh, H., et al. Bone Marrow Transplant, 1998; 22:253-7
Peled et al (Cytotherapy, 2004, 6:344-55

1. A method of selecting a population of ex-vivo expanded hematopoietic stem cells suitable for transplantation, the method comprising:
(a) determining prior to administration in a candidate population of expanded hematopoietic cells at least one of the following parameters:
(i) proportion of CD34+ cells in said population;
(ii) fold expansion of total cells in said population;
(iii) viability of said cells in said population; and
(iv) total number of viable cells; and
(b) selecting or excluding said candidate population according to predetermined values of at least one of said parameters,
thereby selecting a population of ex-vivo expanded hematopoietic stem cells suitable for transplantation.

2. The method of claim 1, wherein said proportion of said CD34+ cells is at least about 4 percent of total cells.

3-4. (canceled)

5. The method of claim 1, wherein said fold expansion of total cells of said population is at least about 10 times.

6-7. (canceled)

8. The method of claim 1, wherein said viability of total cells in said population is at least about 65 percent at 21 days culture.

9-10. (canceled)

11. The method of claim 1, wherein said total number of viable cells of said expanded population is at least about 15×10⁶ viable cells.

12-13. (canceled)

14. The method of claim 1, wherein said selecting is determined according to the values of at least two of said parameters.

15. The method of claim 1, wherein said selecting is according to the values of at least three of said parameters.

16. The method of claim 1, wherein said selecting is according to the values of all four of said parameters.

17. The method of claim 1, wherein said selecting is according to the following values of said parameters:
(i) said proportion of said CD34+ cells being about 4 percent of total cells;
(ii) said fold expansion of total cells of said population is about 50 times;
(iii) said viability of total cells in said population is about 85 percent at 21 days culture; and
(iv) said total number of viable cells of said expanded population is about 23×10⁶ viable cells.

18. The method of claim 1, wherein said hematopoietic stem cells are expanded by propagation ex-vivo by culturing hematopoietic cells in the presence of cytokines and a copper chelator.

19. The method of claim 18, wherein said cytokines are early acting cytokines.

20-22. (canceled)

23. The method of claim 18, wherein said copper chelator is tetrethylenepentamine (TEPA).

24. (canceled)

25. The method of claim 1, wherein said expanded hematopoietic stem cells have been cultured for 21 days.

26. (canceled)

27. The method of claim 1, wherein said hematopoietic cell population is selected from a source consisting of umbilical cord blood, peripheral blood and bone marrow.

28-30. (canceled)

31. The method of claim 27, wherein said hematopoietic cell population has been enriched for CD34+ or CD133+ cells prior to expansion.

32. An expanded hematopoietic stem cell population selected suitable for transplantation according to the method of claim 1.

33. An expanded hematopoietic stem cell population selected suitable for transplantation according to the method of claim 17.

34. A method of treating a hematological disease or condition in a subject in need thereof, the method comprising administering to a subject in need thereof the population of expanded hematopoietic stem cells selected suitable for transplantation of claim 1.

35. A method of treating a hematological disease or condition in a subject in need thereof, the method comprising administering to a subject in need thereof the population of expanded hematopoietic stem cells selected suitable for transplantation of claim 17.

36-43. (canceled)

44. The method of claim 34, wherein said hematological disease is selected from the group consisting of acute myelocytic leukemia (AML), acute lymphocytic leukemia (ALL), chronic lymphocytic leukemia (CLL), chronic myelocytic leukemia (CLL), Hodgkins lymphoma (HL), non-Hodgkins lymphoma (NHL) and myelodysplastic syndrome (MDS).

45-51. (canceled)

52. An article of manufacture comprising a packaging material and a selected ex-vivo expanded hematopoietic cell population, said hematopoietic cell population selected suitable for transplantation by:
(a) determining prior to administration in a candidate population of expanded hematopoietic cells at least one of the following parameters:
(i) proportion of CD34+ cells in said population;
(ii) fold expansion of total cells in said population;
(iii) viability of said cells in said population; and
(iv) total number of viable cells; and
(b) selecting or excluding said candidate population according to predetermined values of at least one of said parameters,
and wherein said packaging material comprises a label or package insert indicating that said hematopoietic cell population is for treating a hematological disease or condition in a subject in need thereof.

53. The article of manufacture of claim 52, wherein said selecting is according to the following values of said parameters:
(i) said proportion of said CD34+ cells being about 4 percent of total cells;
(ii) said fold expansion of total cells of said population is about 50 times;
(iii) said viability of total cells in said population is about 85 percent at 21 days culture; and
(iv)said total number of viable cells of said expanded population is about 23×10⁶ viable cells.
